(19) United States
(12) Patent Application Publication
(10) Pub. No.: US 2024/0382431 A1
(43) Pub. Date: Nov. 21, 2024

(54) Enteric-Coated Particles Containing Lactoferrin

(71) Applicants: UNIVERZA V LJUBLJANI, Ljubljana (SI); ARHEL PROJEKTIRANJE IN INZENIRING D.O.O., Ljubljana-Sentvid (SI)

(72) Inventors: Blaš Grilc, Ljubljana (SI); Maja Bjelosevic, Ljubljana (SI); Robert Roskar, Ljubljana (SI); Nika Osel, Ljubljana (SI); Albin Kristl, Ljubljana (SI); Mirjana Gasperlin, Ljubljana (SI)

(21) Appl. No.: 18/689,731

(22) PCT Filed: Sep. 12, 2022
(86) PCT No.: PCT/EP2022/075283

§ 371 Date: Mar. 6, 2024

(30) Foreign Application Priority Data
Sep. 10, 2021 (LU) LU500645

(51) Int. Cl. A61K 9/50; A61K 38/40

(52) U.S. Cl. CPC A61K 9/5078; A61K 9/5026; A61K 9/5031; A61K 9/5047; A61K 38/40

(57) ABSTRACT

The present invention generally relates to enteric-coated particles containing lactoferrin. More specifically, the present invention provides an enteric-coated particle comprising (or consisting essentially of): a) a core comprising (or consisting essentially of) an inert core-forming material selected from cellulose polymer, sugar, sugar alcohol, starch and carnauba wax; b) a first coating layer substantially covering the core and comprising (or consisting essentially of) b-1) lactoferrin, b-2) a pharmaceutically acceptable binder and optionally b-3) one or more other suitable excipients, such as a plasticizer; and c) a second coating layer substantially covering the first coating layer and comprising (or consisting essentially of) c-1) an enteric coating material, and optionally c-2) one or more suitable excipients, such as a plasticizer and/or an anti-tacking agent. The present invention further provides pharmaceutical compositions and oral dosage forms comprising one or more particles according to the present invention.

### TECHNICAL FIELD OF THE INVENTION

The present invention generally relates to enteric-coated particles containing lactoferrin. More specifically, the present invention provides an enteric-coated particle comprising (or consisting essentially of): a) a core comprising (or consisting essentially of) an inert core-forming material selected from cellulose polymer, sugar, sugar alcohol, starch and carnauba wax; b) a first coating layer substantially covering the core and comprising (or consisting essentially of) b-1) lactoferrin, b-2) a pharmaceutically acceptable binder and optionally b-3) one or more other suitable excipients, such as a plasticizer; and c) a second coating layer substantially covering the first coating layer and comprising (or consisting essentially of) c-1) an enteric coating material, and optionally c-2) one or more suitable excipients, such as a plasticizer and/or an anti-tacking agent. The present invention further provides pharmaceutical compositions and oral dosage forms comprising one or more particles according to the present invention.

### BACKGROUND OF THE INVENTION

Food poisoning is most often treated with antibiotics and/or activated medicinal charcoal. The disadvantage of these approaches is poor selectivity. Antibiotics are also effective against microorganisms that are important for the proper functioning of the gastrointestinal tract. As a result, excessive use of antibiotics can cause constipation, while also increasing the possibility of microorganism mutation to more resistant strains. Therefore, antibiotics are not commonly prescribed for digestive complications unless they are absolutely necessary to prevent dehydration of the patient. Activated charcoal is effective in binding toxins in the gastrointestinal tract and usually does not affect the reproduction of microorganisms. Therefore, its effectiveness in food and water poisoning treatment is difficult to measure.

A solution to the above challenge is the use of a selective microorganism growth inhibition agent to the intestine. In this respect, lactoferrin has been recognized as a protein that inhibits the growth of bacteria and shown to selectively affects only pathogen microorganisms. Lactoferrin (LF) or lactotransferrin is an 80-kDa non-hemic iron-binding globular glycoprotein from the transferrin family, which is found in various secretory fluids. It is the main iron-binding protein in milk with antimicrobial, immunomodulatory, antioxidant, anticancer, and many other biological functions. Due to its potential for the treatment of various diseases, including so-called Traveller's disease, interest in products containing LF is increasing.

On the other hand, lactoferrin generally shows poor low bioavailability after oral administration due to its proteolysis in the gastrointestinal tract and poor permeability across the intestinal epithelia. The half-life of LF in the simulated intestinal fluid is about 3-fold higher than in the simulated gastric fluid. Therefore, the protection of LF against enzymatic degradation and a highly acidic environment in the stomach is crucial for enhancing the stability of LF after oral administration. The most commonly used methods for the delivery of intact LF through the upper gastrointestinal tract are increasing the iron saturation of LF (preparation of hololactoferrin), (micro) encapsulation of LF, and PEGylation. However, these methods have disadvantage(s) in that they decrease the activity of LF, affecting the LF efficacy against microorganisms.

Therefore, there remains a constant need for providing new formulations of lactoferrin which provide advantageous properties such as high dosing and stability of lactoferrin after oral administration.

### SUMMARY OF THE INVENTION

The present invention addresses this need by providing enteric-coated particles comprising: a) a core comprising an inert core-forming material; b) a first coating layer substantially covering the core and comprising b-1) lactoferrin; and c) a second coating layer substantially covering the first coating layer and comprising c-1) an enteric coating material.

More specifically, the present invention thus provides an enteric-coated particle comprising: a) a core comprising an inert core-forming material selected from cellulose polymer, sugar, sugar alcohol, starch and carnauba wax; b) a first coating layer substantially covering the core and comprising b-1) lactoferrin, b-2) a pharmaceutically acceptable binder and optionally b-3) one or more other suitable excipients, such as a plasticizer; and c) a second coating layer substantially covering the first coating layer and comprising c-1) an enteric coating material, and optionally c-2) one or more suitable excipients, such as a plasticizer and/or an anti-tacking agent.

One advantage of the particles of the present invention is that very pure lactoferrin can be provided at a high dose in a single dose. For example, with the particles of the present invention products can be provided which contain 200 mg or more of lactoferrin in a single dose. Moreover, the particles of the present invention provide for improved resistance against conditions found in the gastrointestinal tract, thus adding to the overall stability of lactoferrin after oral administration. The use of the substantially spherical particles according to the present invention may enhance the flow properties, thus filling more uniformly into capsules with less variability and requiring less enteric coating

The present invention may be further characterized by the following items:
1. Enteric-coated particle comprising (or consisting essentially of): a) a core comprising (or consisting essentially of) an inert core-forming material selected from cellulose polymer, sugar, sugar alcohol, starch and carnauba wax; b) a first coating layer substantially covering the core and comprising (or consisting essentially of) b-1) lactoferrin, b-2) a pharmaceutically acceptable binder and optionally b-3) one or more other suitable excipients, such as a plasticizer; and c) a second coating layer substantially covering the first coating layer and comprising (or consisting essentially of) c-1) an enteric coating material, and optionally c-2) one or more suitable excipients, such as a plasticizer and/or an anti-tacking agent.
2. The particle according to item 1, wherein the inert core-forming material is a cellulose polymer.
3. The particle according to item 2, wherein the cellulose polymer is selected from the group consisting of microcrystalline cellulose, hydroxyethylcellulose, hydroxypropylcellulose, hydroxypropylmethylcellulose and carboxymethylcellulose.
4. The particle according to item 2, wherein the cellulose polymer is microcrystalline cellulose.
5. The particle according to item 1, wherein the inert core-forming material is a sugar or sugar alcohol.
6. The particle according to item 5, wherein the sugar is selected from the group consisting of glucose, sucrose and lactose.
7. The particle according to item 5, wherein the sugar alcohol is selected from the group consisting of isomalt, maltitol, mannitol, xylitol and sorbitol.
8. The particle according to any one of items 1 to 7, wherein the pharmaceutically acceptable binder comprised by the first coating layer is selected from the group consisting of cellulose polymers, including hydroxypropylmethylcellulose, hydroxyethylcellulose, hydroxypropylcellulose and carboxymethylcellulose, povidones, including polyvinylpyrrolidone (PVP), copovidones, agar, gelatin, gummi arabicum, alginates, including sodium alginate and polyetylene glycol alginate, polyethylene glycols, polyvinyl alcohols, sugars, sugar alcohols, starches and modified starches including potato starch, maize starch, rice starch, and pre-gelatinised starch.
9. The particle according to any one of items 1 to 8, wherein the pharmaceutically acceptable binder comprised by the first coating layer is a non-polyvinylpyrrolidone (PVP) binder.
10. The particle according to any one of items 1 to 9, wherein the pharmaceutically acceptable binder comprised by the first coating layer is hydroxypropylmethylcellulose.
11. The particle according to any one of items 1 to 10, wherein the first coating layer comprises one or more other suitable excipients, such as a plasticizer.
12. The particle according to any one of items 1 to 11, wherein the first coating layer comprises a plasticizer.
13. The particle according to item 11 or 12, wherein the plasticizer is selected from the group consisting of polyethylene glycols, polyethylene glycol monomethyl ether, propylene glycol, sorbitol-sorbitan solution, glycerin, deacetylated monoglycerides, tributyl citrate, acetyl tributyl citrate, triethyl citrate, acetyl-triethyl-citrate, castor oil, dibutyl sebacate, diethyl phthalate, and triacetin.
14. The particle according to item 11 or 12, wherein the plasticizer is a polyethylene glycol.
15. The particle according to item 14, wherein the polyethylene glycol is selected from polyethylene glycols having an average molecular weight from 1000 g/mol to 20000 g/mol.
16. The particle according to item 14, wherein the polyethylene glycol has an average molecular weight of 6000 g/mol.
17. The particle according to any one of items 1 to 16, wherein the enteric coating material comprised by the second coating layer is selected from polymers or copolymers comprising anionic side groups (preferably carboxylic side groups).
18. The particle according to any one of items 1 to 16, wherein the enteric coating material comprised by the second coating layer is selected from the group consisting of anionic (meth)acrylate copolymers and anionic polyvinyl polymers or copolymers.
19. The particle according to any one of items 1 to 18, wherein the enteric coating material comprised by the second coating layer is an anionic (meth)acrylate copolymer.
20. The particle according to any one of items 1 to 19, wherein the enteric coating material comprised by the second coating layer is an anionic (meth)acrylate copolymer composed of methacrylic acid and methyl methacrylate or ethyl acrylate, or an anionic (meth)acrylate copolymer composed of ethyl acrylate and methyl methacrylate.
21. The particle according to any one of items 1 to 20, wherein the enteric coating material comprised by the second coating layer is an anionic (meth)acrylate copolymer composed of 40 to 60% by weight methacrylic acid and 60 to 40% by weight methyl methacrylate or 60 to 40% by weight ethyl acrylate (e.g., EUDRAGIT® L).
22. The particle according to any one of items 1 to 20, wherein the enteric coating material comprised by the second coating layer is a polymer combination comprising an anionic (meth)acrylate copolymer composed of methacrylic acid and ethyl methacrylate (e.g., EUDRAGIT® L), and an anionic (meth)acrylate copolymer composed of ethyl acrylate and methyl methacrylate (e.g., EUDRAGIT® NM), preferably in the ratio of 25:75.
23. The particle according to any one of items 1 to 18, wherein the enteric coating material comprised by the second coating layer is an anionic polyvinyl polymer or copolymer.
24. The particle according to item 23 wherein the anionic polyvinyl polymers or copolymer may comprise structural units that are derived from unsaturated carboxylic acids other than acrylic acid or methacrylic acid, such as polyvinylacetate-phthalate, a copolymer of vinylacetate and crotonic acid 9:1 or polyvinylacetate-succinate.
25. The particle according to any one of items 1 to 24, wherein the second coating layer further comprises a plasticizer.
26. The particle according to item 25, wherein the plasticizer is selected from the group consisting of polyethylene glycols, polyethylene glycol monomethyl ether, propylene glycol, sorbitol-sorbitan solution, glycerin, deacetylated monoglycerides, tributyl citrate, acetyl tributyl citrate, triethyl citrate, acetyl-triethyl-citrate, castor oil, dibutyl sebacate, diethyl phthalate, and triacetin.
27. The particle according to item 25 or 26, wherein the plasticizer is triethyl citrate (TEC).
28. The particle according to any one of items 1 to 27, wherein the second coating layer further comprises an anti-tacking agent.
29. The particle according to item 28, wherein the anti-tacking agent is talc or glycerol monostearate.
30. The particle according to item 28, wherein the anti-tacking agent is talc.
31. The particle according to item 28, wherein the anti-tacking agent is glycerol monostearate.
32. The particle according to any one of items 1 to 31, wherein the second coating layer further comprises an emulsifier.
33. The particle according to item 32, wherein the emulsifier is a non-ionic emulsifier.
34. The particle according to item 33, wherein the non-ionic emulsifier is a polysorbate.
35. The particle according to item 34, wherein the polysorbate is selected from the group consisting of polyoxyethylene (20) sorbitan monolaurate, polyoxyethylene (20) sorbitan monopalmitate, polyoxyethylene (20) sorbitan monostearate, and polyoxyethylene (20) sorbitan monooleate.
36. The particle according to item 34, wherein the polysorbate is polyoxyethylene (20) sorbitan monooleate.
37. The particle according to item 1, comprising: a) a core comprising microcrystalline cellulose; b) a first coating layer substantially covering the core and comprising b-1) lactoferrin, b-2) hydroxypropylmethylcellulose and b-3) polyethylene glycol; and c) a second coating layer substantially covering the first coating layer and comprising c-1) an anionic (meth)acrylate copolymer, c-2) triethyl citrate (TEC), c-3) glycerol monostearate, and c-4) polyoxyethylene (20) sorbitan monooleate.
38. The particle according to item 1, comprising: a) a core consisting of microcrystalline cellulose; b) a first coating layer substantially covering the core and consisting of a-1) lactoferrin, a-2) hydroxypropylmethylcellulose, a-3) polyethylene glycol, preferably a polyethylene glycol having an average molecular weight of 6000 g/mol, and optionally a-4) impurities; and c) a second coating layer substantially covering the first coating layer and consisting of c-1) an anionic (meth)acrylate copolymer, c-2) triethyl citrate (TEC), c-3) glycerol monostearate, and c-4) polyoxyethylene (20) sorbitan monooleate.
39. The particle according to item 1, comprising: a) a core comprising microcrystalline cellulose; b) a first coating layer substantially covering the core and comprising b-1) lactoferrin, b-2) hydroxypropylmethylcellulose and b-3) polyethylene glycol; and c) a second coating layer substantially covering the first coating layer and comprising c-1) an anionic (meth)acrylate copolymer, c-2) triethyl citrate (TEC) and c-3) talc.
40. The particle according to item 1, comprising: a) a core consisting of microcrystalline cellulose; b) a first coating layer substantially covering the core and consisting of b-1) lactoferrin, b-2) hydroxypropylmethylcellulose, b-3) polyethylene glycol, preferably a polyethylene glycol having an average molecular weight of 6000 g/mol, and optionally b-4) impurities; and c) a second coating layer substantially covering the first coating layer and consisting of c-1) an anionic (meth)acrylate copolymer, c-2) triethyl citrate (TEC) and c-3) talc.
41. The particle according to any one of items 1 to 40, wherein the core comprises about 10% to about 50% of the total weight of the particle.
42. The particle according to any one of items 1 to 40, wherein the core comprises about 20% to about 40% of the total weight of the particle.
43. The particle according to any one of items 1 to 42, wherein the core comprises about 25% to about 35% of the total weight of the particle.
44. The particle according to any one of items 1 to 43, wherein the core comprises about 29% to about 31% of the total weight of the particle.
45. The particle according to any one of items 1 to 44, wherein the first coating layer comprises about 40% to about 60% of the total weight of the particle.
46. The particle according to any one of items 1 to 45, wherein the first coating layer comprises about 45% to about 55% of the total weight of the particle.
47. The particle according to any one of items 1 to 46, wherein the first coating layer comprises about 48% to about 52% of the total weight of the particle.
48. The particle according to any one of items 1 to 47, wherein the second coating layer comprises about 10% to about 30% of the total weight of the particle.
49. The particle according to any one of items 1 to 48, wherein the second coating layer comprises about 15% to about 25% of the total weight of the particle.
50. The particle according to any one of items 1 to 49, wherein the second coating layer comprises about 18% to about 22% of the total weight of the particle.
51. The particle according to any one of items 1 to 50, wherein the first coating layer comprises lactoferrin in an amount of about 70% to about 90% of the total weight of the first coating layer.
52. The particle according to any one of items 1 to 51, wherein the first coating layer comprises lactoferrin in an amount of about 75% to about 85% of the total weight of the first coating layer.
53. The particle according to any one of items 1 to 52, wherein the first coating layer comprises lactoferrin in an amount of about 76% to about 80% of the total weight of the first coating layer.
54. The particle according to any one of items 1 to 53, wherein the first coating layer comprises the pharmaceutically acceptable binder in an amount of about 7% to about 30% of the total weight of the first coating layer.
55. The particle according to any one of items 1 to 54, wherein the first coating layer comprises the pharmaceutically acceptable binder in an amount of about 10% to about 25% of the total weight of the first coating layer.
56. The particle according to any one of items 1 to 55, wherein the first coating layer comprises the pharmaceutically acceptable binder in an amount of about 10% to about 20% of the total weight of the first coating layer.
57. The particle according to any one of items 1 to 56, wherein the first coating layer comprises the polymeric binder in an amount of about 10% to about 15% of the total weight of the first coating layer.
58. The particle according to any one of items 1 to 57 wherein the first coating layer comprises one or more other suitable excipients, such as a plasticizer, in an amount of about 0,1% to about 5% of the total weight of the first coating layer.
59. The particle according to any one of items 1 to 58, wherein the first coating layer comprises the plasticizer in an amount of about 1% to about 5% of the total weight of the first coating layer.
60. The particle according to any one of items 1 to 59, wherein the first coating layer comprises the plasticizer in an amount of about 2% to about 4% of the total weight of the first coating layer.
61. The particle according to any one of items 1 to 60, wherein the second coating layer comprises the enteric coating material in an amount of about 50% to about 90% of the total weight of the second coating layer.
62. The particle according to any one of items 1 to 61, wherein the second coating layer comprises the enteric coating material in an amount of about 60% to about 90% of the total weight of the second coating layer.
63. The particle according to any one of items 1 to 62, wherein the second coating layer comprises the enteric coating material in an amount of about 60% to about 70% of the total weight of the second coating layer.
64. The particle according to any one of items 1 to 62, wherein the second coating layer comprises the enteric coating material in an amount of about 60% to about 65% of the total weight of the second coating layer.
65. The particle according to any one of items 1 to 62, wherein the second coating layer comprises the enteric coating material in an amount of about 70% to about 90% of the total weight of the second coating layer.
66. The particle according to any one of items 1 to 62, wherein the second coating layer comprises the enteric coating material in an amount of about 80% to about 90% of the total weight of the second coating layer.
67. The particle according to any one of items 1 to 66, wherein the second coating layer comprises one or more other suitable excipients, such as a plasticizer, in a total amount of about 5% to about 20% of the total weight of the second coating layer.
68. The particle according to any one of items 1 to 67, wherein the second coating layer comprises one or more other suitable excipients, such as a plasticizer, in a total amount of about 10% to about 20% of the total weight of the second coating layer.
69. The particle according to any one of items 1 to 68, wherein the second coating layer comprises the plasticizer in an amount of about 5% to about 10% of the total weight of the second coating layer.
70. The particle according to any one of items 1 to 69, wherein the second coating layer comprises the anti-tacking agent in an amount of about 2% to about 35% of the total weight of the second coating layer.
71. The particle according to any one of items 1 to 70, wherein the second coating layer comprises glycerol monostearate as the anti-tacking agent in an amount of about 3% to about 5% of the total weight of the second coating layer, such as in an amount of about 4% to about 4,5% of the total weight of the second coating layer.
72. The particle according to any one of items 1 to 71, wherein the second coating layer comprises talc as the anti-tacking agent in an amount of about 25% to about 35% of the total weight of the second coating layer, such as in an amount of about 28% to about 32% of the total weight of the second coating layer.
73. The particle according to any one of items 1 to 72, wherein the second coating layer comprises the emulsifier in an amount of about 1% to about 3% of the total weight of the second coating layer.
74. The particle according to any one of items 1 to 73, wherein lactoferrin is present in an amount of about 30% to about 50% of the total weight of the particle.
75. The particle according to any one of items 1 to 74, wherein lactoferrin is present in an amount of about 35% to about 45% of the total weight of the particle.
76. The particle according to any one of items 1 to 75, wherein lactoferrin is present in an amount of about 35% to about 40% of the total weight of the particle.
77. The particle according to any one of items 1 to 76, wherein the pharmaceutically acceptable binder comprised by the first coating layer is present in an amount of about 5% to about 10% of the total weight of the particle.
78. The particle according to any one of items 1 to 77, wherein the pharmaceutically acceptable binder comprised by the first coating layer is present in an amount of about 5% to about 8% of the total weight of the particle.
79. The particle according to any one of items 1 to 77, wherein the pharmaceutically acceptable binder comprised by the first coating layer is present in an amount of about 6% to about 7% of the total weight of the particle.
80. The particle according to any one of items 1 to 79, wherein the plasticizer, if comprised by the first coating layer, is present in an amount of about 1% to about 2% of the total weight of the particle.
81. The particle according to any one of items 1 to 80, wherein the plasticizer, if comprised by the first coating layer, is present in an amount of about 1% to about 1,5% of the total weight of the particle.
82. The particle according to any one of items 1 to 81, wherein the enteric coating material comprised by the second coating layer is present in an amount of about 15% to about 20% of the total weight of the particle.
83. The particle according to any one of items 1 to 82, wherein the enteric coating material comprised by the second coating layer is present in an amount of about 16% to about 18% of the total weight of the particle.
84. The particle according to any one of items 1 to 83, wherein the plasticizer, if comprised by the second coating layer, is present in an amount of about 1% to about 2% of the total weight of the particle.
85. The particle according to any one of items 1 to 84, wherein the anti-tacking agent, if comprised by the second coating layer, is present in an amount of about 0.5% to about 10% of the total weight of the particle.
86. The particle according to any one of items 1 to 85, wherein glycerol monostearate, if comprised by the second coating layer as the anti-tacking agent, is present in an amount of about 0.5% to about 1% of the total weight of the particle.
87. The particle according to any one of items 1 to 86, wherein the emulsifier, if comprised by the second coating layer, is present in an amount of about 0.1% to about 1% of the total weight of the particle.
88. The particle according to any one of items 1 to 87, wherein the emulsifier, if comprised by the second coating layer, is present in an amount of about 0.1% to about 0.5% of the total weight of the particle.
89. The particle according to any one of items 1 to 88, which is in the form of a pellet, spheroid or bead.
90. The particle according to any one of items 1 to 88, which is in the form of a pellet.
91. The particle according to any one of items 1 to 90, wherein said particle has a mean particle size d50 of about 450 μm to about 650 μm.
92. The particle according to any one of items 1 to 91, wherein said particle has a bulk density of about 0.7 g/ml to about 0.8 g/ml, such as about 0.740 g/mL.
93. The particle according to any one of items 1 to 92, wherein said particle has a tapped density of about 0.75 g/ml to about 0.85 g/ml, such as about 0.802 g/mL.
94. The particle according to any one of items 1 to 93, wherein said particle is substantially spherical
95. The particle according to any one of items 1 to 94, wherein at most 10% of lactoferrin is released from the particle within 120 minutes as measured by an in vitro dissolution test according to Ph. Eur. 2.9.3.
96. Pharmaceutical composition comprising one or more particles according to any one of items 1 to 95, and optionally one or more pharmaceutically acceptable excipients.
97. Oral dosage form comprising one or more particles according to any one of items 1 to 95.
98. Oral dosage form according to item 97, which is a solid dosage form, such as capsule or tablet.
99. Oral dosage form according to item 97 or 98, which is a capsule.
100. Oral dosage form according to any one of items 97 to 99, which is a hard capsule.
101. The oral dosage form according to any of items 97 to 100, which comprises 200 mg or more of lactoferrin.
102. The oral dosage form according to any of items 97 to 101, which comprises from about 400 mg to about 600 mg of said particles.
103. The oral dosage form according to any of items 97 to 102, which comprises at least 500 mg of said particles.
104. The particle according to any one of items 1 to 95, the pharmaceutical composition according to item 96 or the oral dosage form according to any one of items 97 to 103 for use as a medicament.
105. The particle according to any one of items 1 to 95, the pharmaceutical composition according to item 96 or the oral dosage form according to any one of items 97 to 103 for use in the prophylaxis and/or treatment of an intestinal disorder.
106. The particle according to any one of items 1 to 95, the pharmaceutical composition according to item 96 or the oral dosage form according to any one of items 97 to 103 for use in the prophylaxis and/or treatment of Traveler's diarrhea.
107. The particle according to any one of items 1 to 95, the pharmaceutical composition according to item 96 or the oral dosage form according to any one of items 97 to 103 for use in the regulation of the healthy balance of human intestine microbiota.
108. The particle according to any one of items 1 to 95, the pharmaceutical composition according to item 96 or the oral dosage form according to any one of items 97 to 103 for use in the inhibition of microorganism growth in the human intestine.
109. Food or food supplement comprising one or more particles according to any one of items 1 to 95.
110. Food or food supplement according to item 109 for balancing the ratio between a pathogen and the beneficial microorganisms in the human intestine.
111. Process for producing the particle according to any one of items 1 to 95, comprising applying the first coating layer in the form of an aqueous coating solution, suspension or dispersion in a spray procedure or by fluidized bed spray granulation onto the core, and applying the second coating layer in the form of an aqueous coating solution, suspension or dispersion in a spray procedure or by fluidized bed spray granulation onto the first coating layer.
112. The process according to item 111, comprising forming the core by direct compression, wet or dry granulation, or direct pelleting of the core-forming material.
113. Process for producing the oral dosage form according to any one of items 97 to 103, comprising filling one or more particles according to any one of claims 1 to 93 into a capsule.

### BRIEF DESCRIPTION OF THE FIGURES

FIG. 1: Dissolution profile of the final product (USP II).

FIG. 2: Particle size distribution of the final product.

The present invention is now described in more detail below.

### DETAILED DESCRIPTION OF THE INVENTION

As noted above, the present invention is based on the finding that formulating lactoferrin as enteric-coated, multi-layered particles allows for a high dose of very pure lactoferrin in a single dose. For example, with the particles of the present invention products can be provided which contain 200 mg or more of lactoferrin in a single dose. Moreover, the particles of the present invention provide for improved resistance against conditions found in the gastrointestinal tract, thus adding to the overall stability of lactoferrin after oral administration.

The present invention thus provides an enteric-coated particle comprising (or consisting essentially of): a) a core comprising (or consisting essentially of) cellulose polymer, sugar, sugar alcohol, starch and carnauba wax; b) a first coating layer substantially covering the core and comprising (or consisting essentially of) b-1) lactoferrin, b-2) a pharmaceutically acceptable binder and optionally b-3) one or more other suitable excipients, such as a plasticizer; and c) a second coating layer substantially covering the first coating layer and comprising (or consisting essentially of) c-1) an enteric coating material, and optionally c-2) one or more suitable excipients, such as a plasticizer and/or an anti-tacking agent.

According to some embodiments, the inert core-forming material is a cellulose polymer, such as a cellulose polymer selected from the group consisting of microcrystalline cellulose, hydroxyethylcellulose, hydroxypropylcellulose, hydroxypropylmethylcellulose and carboxymethylcellulose. According to some embodiments, the cellulose polymer is microcrystalline cellulose.

According to some embodiments, the inert core-forming material is a sugar or sugar alcohol.

According to some embodiments, the inert core-forming material is a sugar, such as a sugar selected from the group consisting of glucose, sucrose and lactose.

According to some embodiments, the inert core-forming material is a sugar alcohol, such as a sugar alcohol selected from the group consisting of isomalt, maltitol, mannitol, xylitol and sorbitol.

According to some embodiments, the inert core-forming material is starch.

According to some embodiments, the inert core-forming material is carnauba wax.

The inert core may be obtained commercially. Non-limiting examples of commercially available beads or pellets to be used as the core for the particles disclosed herein include sugar spheres (for example, Paular spheres), Cellets® cores, such as Cellets® 100, Cellets® 200, Cellets® 350, Cellets® 500, Cellets® 700, or Cellets® 1000 (HARKE Group, Mülheim am der Ruhr, Germany), carnauba wax cores such as C-Wax Pellets® or mannitol cores such as M-Cell®. Alternatively, the core may be prepared de novo using a variety of well-known granulation methods, including high sheer wet granulation, spray drying, and fluid bed granulation (including rotary fluid bed granulation) or by direct compression or direct pelleting of the core-forming material.

According to some embodiments, the core comprises about 10% to about 50% of the total weight of the particle. According to some embodiments, the core comprises about 20% to about 40% of the total weight of the particle. According to some embodiments, the core comprises about 25% to about 35% of the total weight of the particle. According to some embodiments, the core comprises about 29% to about 31% of the total weight of the particle.

The first coating layer substantially covering the core generally comprises lactoferrin (as the active ingredient), a pharmaceutically acceptable binder and optionally one or more other suitable excipients.

According to some embodiments, the first coating layer comprises about 40% to about 60% of the total weight of the particle. According to some embodiments, the first coating layer comprises about 45% to about 55% of the total weight of the particle. According to some embodiments, the first coating layer comprises about 48% to about 52% of the total weight of the particle. According to some embodiments, the second coating layer comprises about 10% to about 30% of the total weight of the particle. According to some embodiments, the second coating layer comprises about 15% to about 25% of the total weight of the particle. According to some embodiments, the second coating layer comprises about 18% to about 22% of the total weight of the particle.

Lactoferrin (LF), also known as lactotransferrin (LTF), is used as the active ingredient within the context of the present invention. As note above, it is an 80-kDa non-hemic iron-binding globular glycoprotein from the transferrin family, which is found in various secretory fluids such as milk, saliva, tears, and nasal secretions. It is the main iron-binding protein in milk with antimicrobial, immunomodulatory, antioxidant, anticancer, and many other biological functions. Human colostrum (“first milk”) has the highest concentration, followed by human milk, then cow milk (150 mg/L). At least 60 gene sequences of lactoferrin have been characterized in 11 species of mammals. Lactoferrin may be obtained commercially. Alternatively, it may be purified from milk (notably cow's milk) or produced recombinantly. Lactoferrin may be employed in pure form or as a preparation (e.g., extract) containing at least 90% (such as at least 93%) by weight of Lactoferrin. Depending on the method of preparation, the obtained lactoferrin containing preparation may contain some residual impurities, which eventually may also be present in the first coating layer, but do not materially affect the basic and novel characteristic(s) of the particle of the present invention.

According to some embodiments, the first coating layer comprises lactoferrin in an amount of about 70% to about 90% of the total weight of the first coating layer. According to some embodiments, the first coating layer comprises lactoferrin in an amount of about 75% to about 85% of the total weight of the first coating layer. According to some embodiments, the first coating layer comprises lactoferrin in an amount of about 75% to about 82% of the total weight of the first coating layer. According to some embodiments, the first coating layer comprises lactoferrin in an amount of about 76% to about 80% of the total weight of the first coating layer, such as about 78% of the total weight of the first coating layer.

According to some embodiments, lactoferrin is present in an amount of about 30% to about 50% of the total weight of the particle. According to some embodiments, lactoferrin is present in an amount of about 35% to about 45% of the total weight of the particle. According to some embodiments, lactoferrin is present in an amount of about 35% to about 40% of the total weight of the particle.

The pharmaceutically acceptable binder may for example be a cellulose polymer, such as hydroxypropylmethylcellulose, hydroxyethylcellulose, hydroxypropylcellulose and carboxymethylcellulose, a povidone, such as polyvinylpyrrolidone (PVP), a copovidone, agar, gelatin, gummi arabicum, an alginate, such as sodium alginate and polyetylene glycol alginate, a polyethylene glycol, a polyvinyl alcohol, a sugar, a sugar alcohol, a starch or modified starch, such as potato starch, maize starch or rice starch, or pre-gelatinised starch.

According to some embodiments, the pharmaceutically acceptable binder comprised by the first coating layer is selected from the group consisting of cellulose polymers, including hydroxypropylmethylcellulose, hydroxyethylcellulose, hydroxypropylcellulose and carboxymethylcellulose, povidones, including polyvinylpyrrolidone (PVP), copovidones, agar, gelatin, gummi arabicum, alginates, including sodium alginate and polyetylene glycol alginate, polyethylene glycols, polyvinyl alcohols, sugars, sugar alcohols, starches and modified starches including potato starch, maize starch, rice starch, and pre-gelatinised starch.

According to some embodiments, the pharmaceutically acceptable binder comprised by the first coating layer is hydroxypropylmethylcellulose.

According to some embodiments, the first coating layer comprises the pharmaceutically acceptable binder in an amount of about 7% to about 30% of the total weight of the first coating layer. According to some embodiments, the first coating layer comprises the pharmaceutically acceptable binder in an amount of about 10% to about 25% of the total weight of the first coating layer. According to some embodiments, the first coating layer comprises the pharmaceutically acceptable binder in an amount of about 10% to about 20% of the total weight of the first coating layer. According to some embodiments, the first coating layer comprises the pharmaceutically acceptable binder in an amount of about 10% to about 15% of the total weight of the first coating layer.

According to some embodiments, the pharmaceutically acceptable binder comprised by the first coating layer is present in an amount of about 5% to about 10% of the total weight of the particle. According to some embodiments, the pharmaceutically acceptable binder comprised by the first coating layer is present in an amount of about 5% to about 8% of the total weight of the particle. According to some embodiments, the pharmaceutically acceptable binder comprised by the first coating layer is present in an amount of about 6% to about 7% of the total weight of the particle.

According to some embodiments, the first coating layer comprises one or more other suitable excipients, such as a plasticizer.

According to some embodiments, the first coating layer comprises a plasticizer.

The plasticizer may for example be a polyethylene glycol, polyethylene glycol monomethyl ether, propylene glycol, sorbitol-sorbitan solution, glycerin, deacetylated monoglycerides, tributyl citrate, acetyl tributyl citrate, triethyl citrate, acetyl-triethyl-citrate, castor oil, dibutyl sebacate, diethyl phthalate, or triacetin.

According to some embodiments, the first coating layer comprises a plasticizer selected from the group consisting of polyethylene glycols, polyethylene glycol monomethyl ether, propylene glycol, sorbitol-sorbitan solution, glycerin, deacetylated monoglycerides, tributyl citrate, acetyl tributyl citrate, triethyl citrate, acetyl-triethyl-citrate, castor oil, dibutyl sebacate, diethyl phthalate, and triacetin.

According to some embodiments, the plasticizer is a polyethylene glycol, such as a polyethylene glycol selected from polyethylene glycols having an average molecular weight from 1000 g/mol to 20000 g/mol.

According to some embodiments, the plasticizer is a polyethylene glycol having an average molecular weight from 1000 g/mol to 10000 g/mol.

According to some embodiments, the plasticizer is a polyethylene glycol having an average molecular weight from 4000 g/mol to 8000 g/mol.

According to some embodiments, the plasticizer is a polyethylene glycol having an average molecular weight of 6000 g/mol.

According to some embodiments, the first coating layer comprises one or more other suitable excipients, such as a plasticizer, in an amount of about 0.1% to about 5% of the total weight of the first coating layer. According to some embodiments, the first coating layer comprises the plasticizer in an amount of about 1% to about 5% of the total weight of the first coating layer. According to some embodiments, the first coating layer comprises the plasticizer in an amount of about 2% to about 4% of the total weight of the first coating layer.

According to some embodiments, the plasticizer, if comprised by the first coating layer, is present in an amount of about 1% to about 2% of the total weight of the particle. According to some embodiments, the plasticizer, if comprised by the first coating layer, is present in an amount of about 1% to about 1.5% of the total weight of the particle.

The second coating layer substantially covering the first coating layer generally functions as an enteric coating, which means that it prevents the dissolution or disintegration of the particle in the gastric environment, thereby protecting lactoferrin from the acidity of the stomach. To this end, the second coating layer comprises an enteric coating material.

Suitable enteric coating materials are well-known to the skilled person, and include, but are not limited to, polymers or copolymers comprising anionic side groups (preferably carboxylic side groups), such as methyl methacrylate-methacrylic acid copolymers. Other non-limiting examples of enteric coating materials are cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), shellac, cellulose acetate trimellitate, sodium alginate or zein.

According to some embodiments, the enteric coating material comprised by the second coating layer is selected from polymers or copolymers comprising anionic side groups (preferably carboxylic side groups).

According to some embodiments, the enteric coating material comprised by the second coating layer is selected from the group consisting of anionic (meth)acrylate copolymers and anionic polyvinyl polymers or copolymers.

According to some embodiments, the enteric coating material comprised by the second coating layer is an anionic (meth)acrylate copolymer.

Non-limiting examples of anionic (meth)acrylate copolymer can include ammonio methacrylate copolymer, basic butylated methacrylate copolymer, methacrylic acid-methyl methacrylate copolymer (1:1), methacrylic acid-ethyl acrylate copolymer (1:1), methacrylic acid-ethyl acrylate copolymer (1:1), methacrylic acid-methyl methacrylate copolymer (1:2), polyacrylate dispersion 30%, methacrylic acid copolymer, amino methacrylate copolymer, ammonio methacrylate copolymer, ammonio methacrylate copolymer dispersion, ethyl acrylate and methyl methacrylate copolymer, and combinations thereof. Some anionic (meth)acrylate copolymers are available commercially as Eudragit® E 12.5, Eudragit® E 100, Eudragit® E PO, Eudragit® L 12.5 P, Eudragit® L 12.5, Eudragit® L 100, Eudragit® L 100-55, Eudragit® L 30 D-55, Eudragit® S 12.5 P, Eudragit® S 12.5, Eudragit® S 100, Eudragit® FS 30 D, Eudragit® RL 12.5, Eudragit® RL 100, Eudragit® RL PO, Eudragit® RL 30 D, Eudragit® RS 12.5, Eudragit® RS 100, Eudragit® RS PO, Eudragit® RS 30 D, Eudragit® NE 30 D, Eudragit® NE 40 D, Eudragit® NM 30 D, Eastacryl™ 30 D, Kollicoat® MAE 30 DP, Kollicoat® MAE 100 P, Acryl-EZE®, Acryl-EZE® 93 A, and Acryl-EZE® MP.

According to some embodiments, the enteric coating material comprised by the second coating layer is an anionic (meth)acrylate copolymer composed of methacrylic acid and methyl methacrylate or ethyl acrylate.

According to some embodiments, the enteric coating material comprised by the second coating layer is an anionic (meth)acrylate copolymer composed of methacrylic acid and methyl methacrylate.

According to some embodiments, the enteric coating material comprised by the second coating layer is an anionic (meth)acrylate copolymer composed of methacrylic acid and ethyl acrylate.

A (meth)acrylate monomer having an anionic group is, for example, acrylic acid, with a preference for methacrylic acid. Suitable anionic (meth)acrylate copolymers are those composed of 40 to 60% by weight methacrylic acid and 60 to 40% by weight methyl methacrylate or 60 to 40% by weight ethyl acrylate (e.g., EUDRAGIT® L or EUDRAGIT® L 100-55 types). EUDRAGIT® L is a copolymer of 50% by weight methyl methacrylate and 50% by weight methacrylic acid. EUDRAGIT® L 100-55 is a copolymer of 50% by weight ethyl acrylate and 50% by weight methacrylic acid. EUDRAGIT® L 30 D-55 is a dispersion comprising 30% by weight EUDRAGIT® L 100-55. Likewise suitable are anionic (meth)acrylate copolymers composed of 20 to 40% by weight methacrylic acid and 80 to 60% by weight methyl methacrylate (EUDRAGIT® S type). Suitable (meth)acrylate copolymers are those consisting of 10 to 30% by weight methyl methacrylate, 50 to 70% by weight methyl acrylate and 5 to 15% by weight methacrylic acid (e.g., EUDRAGIT® FS type). EUDRAGIT® FS is a copolymer of 25% by weight methyl methacrylate, 65% by weight methyl acrylate and 10% by weight methacrylic acid. EUDRAGIT® FS 30 D is a dispersion comprising 30% by weight EUDRAGIT® FS.

According to some embodiments, the enteric coating material comprised by the second coating layer is an anionic (meth)acrylate copolymer composed of 40 to 60% by weight methacrylic acid and 60 to 40% by weight methyl methacrylate or 60 to 40% by weight ethyl acrylate.

According to some embodiments, the enteric coating material comprised by the second coating layer is an anionic (meth)acrylate copolymer composed of 50% by weight methacrylic acid and 50% by weight methyl methacrylate.

According to some embodiments, the enteric coating material comprised by the second coating layer is an anionic (meth)acrylate copolymer composed of ethyl acrylate and methyl methacrylate.

According to some embodiments, the enteric coating material comprised by the second coating layer is an anionic (meth)acrylate copolymer composed of 40 to 60% by weight ethyl acrylate and 60 to 40% by weight methyl methacrylate (e.g., EUDRAGIT® NM).

According to some embodiments, the enteric coating material comprised by the second coating layer is a polymer combination comprising an anionic (meth)acrylate copolymer composed of methacrylic acid and ethyl methacrylate (e.g., EUDRAGIT® L), and an anionic (meth)acrylate copolymer composed of ethyl acrylate and methyl methacrylate (e.g., EUDRAGIT® NM), preferably in the ratio of 25:75.

According to some embodiments, the enteric coating material comprised by the second coating layer is a polymer combination comprising an anionic (meth)acrylate copolymer composed of 40 to 60% by weight methacrylic acid and 60 to 40% by weight ethyl methacrylate (e.g., EUDRAGIT® L), and an anionic (meth)acrylate copolymer composed of 40 to 60% by weight ethyl acrylate and 60 to 40% by weight methyl methacrylate (e.g., EUDRAGIT® NM), preferably in the ratio of 25:75.

According to some embodiments, the enteric coating material comprised by the second coating layer is an anionic polyvinyl polymer or copolymer. Such anionic polyvinyl polymers or copolymer may comprise structural units that are derived from unsaturated carboxylic acids other than acrylic acid or methacrylic acid, such as polyvinylacetate-phthalate, a copolymer of vinylacetate and crotonic acid 9:1 or polyvinylacetate-succinate.

According to some embodiments, the second coating layer comprises the enteric coating material in an amount of about 50% to about 90% of the total weight of the second coating layer. According to some embodiments, the second coating layer comprises the enteric coating material in an amount of about 60% to about 90% of the total weight of the second coating layer. According to some embodiments, the second coating layer comprises the enteric coating material in an amount of about 60% to about 70% of the total weight of the second coating layer. According to some embodiments, the second coating layer comprises the enteric coating material in an amount of about 60% to about 65% of the total weight of the second coating layer. According to some embodiments, the second coating layer comprises the enteric coating material in an amount of about 70% to about 90% of the total weight of the second coating layer. According to some embodiments, the second coating layer comprises the enteric coating material in an amount of about 80% to about 90% of the total weight of the second coating layer.

According to some embodiments, the enteric coating material comprised by the second coating layer is present in an amount of about 15% to about 20% of the total weight of the particle. According to some embodiments, the enteric coating material comprised by the second coating layer is present in an amount of about 16% to about 18% of the total weight of the particle.

The second coating layer may further comprise one or more suitable excipients, such as a plasticizer and/or an anti-tacking agent.

According to some embodiments, the second coating layer further comprises a plasticizer, such as a plasticizer is selected from the group consisting of polyethylene glycols, polyethylene glycol monomethyl ether, propylene glycol, sorbitol-sorbitan solution, glycerin, deacetylated monoglycerides, tributyl citrate, acetyl tributyl citrate, triethyl citrate, acetyl-triethyl-citrate, castor oil, dibutyl sebacate, diethyl phthalate, and triacetin.

According to some embodiments, the plasticizer comprised by the second coating layer is triethyl citrate (TEC).

According to some embodiments, the plasticizer comprised by the second coating layer is a polyethylene glycol, such as a polyethylene glycol selected from polyethylene glycols having an average molecular weight from 200 g/mol to 15000 g/mol.

According to some embodiments, the plasticizer comprised by the second coating layer is a polyethylene glycol having an average molecular weight from 200 g/mol to 6000 g/mol.

According to some embodiments, the plasticizer comprised by the second coating layer is a polyethylene glycol having an average molecular weight from 200 g/mol to 800 g/mol.

According to some embodiments, the plasticizer comprised by the second coating layer is a polyethylene glycol having an average molecular weight of 400 g/mol.

According to some embodiments, the second coating layer comprises one or more other suitable excipients, such as a plasticizer, in a total amount of about 5% to about 20% of the total weight of the second coating layer. According to some embodiments, the second coating layer comprises one or more other suitable excipients, such as a plasticizer, in a total amount of about 10% to about 20% of the total weight of the second coating layer.

According to some embodiments, the second coating layer comprises the plasticizer in an amount of about 5% to about 10% of the total weight of the second coating layer.

According to some embodiments, the plasticizer, if comprised by the second coating layer, is present in an amount of about 1% to about 2% of the total weight of the particle.

According to some embodiments, the second coating layer further comprises an anti-tacking agent, such as talc or glycerol monostearate.

According to some embodiments, the anti-tacking agent is talc. According to some embodiments, the anti-tacking agent is glycerol monostearate.

According to some embodiments, the second coating layer comprises the anti-tacking agent in an amount of about 2% to about 35% of the total weight of the second coating layer.

According to some embodiments, the anti-tacking agent, if comprised by the second coating layer, is present in an amount of about 0.5% to about 10% of the total weight of the particle.

According to some embodiments, the second coating layer comprises glycerol monostearate as the anti-tacking agent in an amount of about 3% to about 5% of the total weight of the second coating layer, such as in an amount of about 4% to about 4.5% of the total weight of the second coating layer.

According to some embodiments, glycerol monostearate, if comprised by the second coating layer as the anti-tacking agent, is present in an amount of about 0.5% to about 1% of the total weight of the particle.

According to some embodiments, the second coating layer comprises talc as the anti-tacking agent in an amount of about 25% to about 35% of the total weight of the second coating layer, such as in an amount of about 28% to about 32% of the total weight of the second coating layer.

According to some embodiments, talc, if comprised by the second coating layer as the anti-tacking agent, is present in an amount of about 8% to about 12% of the total weight of the particle.

According to some embodiments, the second coating layer further comprises an emulsifier.

According to some embodiments, the emulsifier is a non-ionic emulsifier, such as a polysorbate.

According to some embodiments, the non-ionic emulsifier is a polysorbate selected from the group consisting of polyoxyethylene (20) sorbitan monolaurate, polyoxyethylene (20) sorbitan monopalmitate, polyoxyethylene (20) sorbitan monostearate, and polyoxyethylene (20) sorbitan monooleate. According to some embodiments, the non-ionic emulsifier is polyoxyethylene (20) sorbitan monooleate.

According to some embodiments, the second coating layer comprises the emulsifier in an amount of about 1% to about 3% of the total weight of the second coating layer. According to some embodiments, the second coating layer comprises the emulsifier in an amount of about 1% to about 2% of the total weight of the second coating layer.

According to some embodiments, the emulsifier, if comprised by the second coating layer, is present in an amount of about 0.1% to about 1% of the total weight of the particle. According to some embodiments, the emulsifier, if comprised by the second coating layer, is present in an amount of about 0.1% to about 0.5% of the total weight of the particle.

Depending on the material and method used in the preparation of the particle, the particle of the present invention or any of its layers may comprise impurities and/or water in the form of, e.g., moisture, which do not materially affect the basic and novel characteristic(s) of the particle of the present invention. Generally, if present, the impurities will not make out more than about 5% of the total weight of the particle, preferably will not make out more than about 3% of the total weight of the particle. Specifically, if present, the impurities will not make out more than about 10% of the total weight of the first coating layer, preferably will not make out more than about 8.5% of the total weight of the particle. Likewise, if present, water in the form of, e.g., moisture, will not make out more than about 7% of the total weight of the particle, preferably will not make out more than about 5% of the total weight of the particle. Specifically, if present, water in the form of, e.g., moisture, will not make out more than about 7% of the total weight of the first coating layer and/or second coating layer, preferably will not make out more than about 5% of the first coating layer and/or second coating layer.

According to certain embodiments, the particle comprises: a) a core comprising microcrystalline cellulose; b) a first coating layer substantially covering the core and comprising b-1) lactoferrin, b-2) hydroxypropylmethylcellulose and b-3) polyethylene glycol; and c) a second coating layer substantially covering the first coating layer and comprising c-1) an anionic (meth)acrylate copolymer, c-2) triethyl citrate (TEC), c-3) glycerol monostearate, and c-4) polyoxyethylene (20) sorbitan monooleate.

According to certain embodiments, the particle comprises (or consists essentially of): a) a core consisting essentially of microcrystalline cellulose; b) a first coating layer substantially covering the core and consisting essentially of b-1) lactoferrin, b-2) hydroxypropylmethylcellulose and b-3) polyethylene glycol; and c) a second coating layer substantially covering the first coating layer and consisting essentially of c-1) an anionic (meth)acrylate copolymer, c-2) triethyl citrate (TEC), c-3) glycerol monostearate, and c-4) polyoxyethylene (20) sorbitan monooleate.

According to certain embodiments, the particle comprises: a) a core comprising microcrystalline cellulose; b) a first coating layer substantially covering the core and comprising b-1) lactoferrin, b-2) hydroxypropylmethylcellulose and b-3) polyethylene glycol; and c) a second coating layer substantially covering the first coating layer and comprising c-1) an anionic (meth)acrylate copolymer, c-2) triethyl citrate (TEC) and c-3) talc.

According to certain embodiments, the particle comprises (or consists essentially of): a) a core consisting essentially of microcrystalline cellulose; b) a first coating layer substantially covering the core and consisting essentially of b-1) lactoferrin, b-2) hydroxypropylmethylcellulose and b-3) polyethylene glycol; and c) a second coating layer substantially covering the first coating layer and consisting essentially of c-1) an anionic (meth)acrylate copolymer, c-2) triethyl citrate (TEC) and c-3) talc.

According to certain embodiments, the particle comprises (or consists essentially of): a) a core comprising microcrystalline cellulose, said core comprises about 25% to about 35% of the total weight of the particle; b) a first coating layer substantially covering the core and comprising b-1) lactoferrin in an amount of about 30% to about 50% of the total weight of the particle such as in an amount of about 35% to about 45% of the total weight of the particle, b-2) hydroxypropylmethylcellulose in an amount of about 5% to about 10% of the total weight of the particle, such as in an amount of about 5% to about 8% of the total weight of the particle, and b-3) polyethylene glycol (such as polyethylene glycol having an average molecular weight of 6000 g/mol) in an amount of about 1% to about 2% of the total weight of the particle, such as in an amount of about 1% to about 1.5% of the total weight of the particle; and c) a second coating layer substantially covering the first coating layer and comprising c-1) an anionic (meth)acrylate copolymer in an amount of about 15% to about 20% of the total weight of the particle, such as in an amount of about 16% to about 18% of the total weight of the particle, c-2) triethyl citrate (TEC) in an amount of about 1% to about 2% of the total weight of the particle, c-3) glycerol monostearate in an amount of about 0.5% to about 1% of the total weight of the particle, and c-4) polyoxyethylene (20) sorbitan monooleate in an amount of about 0.1% to about 1% of the total weight of the particle, wherein the sum of all ingredients adds up to 100% of the total weight of the particle.

According to certain embodiments, the particle comprises (or consists essentially of): a) a core consisting essentially of microcrystalline cellulose, said core comprises about 25% to about 35% of the total weight of the particle; b) a first coating layer substantially covering the core and consisting essentially of b-1) lactoferrin in an amount of about 30% to about 50% of the total weight of the particle such as in an amount of about 35% to about 45% of the total weight of the particle, b-2) hydroxypropylmethylcellulose in an amount of about 5% to about 10% of the total weight of the particle, such as in an amount of about 5% to about 8% of the total weight of the particle, and b-3) polyethylene glycol (such as polyethylene glycol having an average molecular weight of 6000 g/mol) in an amount of about 1% to about 2% of the total weight of the particle, such as in an amount of about 1% to about 1.5% of the total weight of the particle; and c) a second coating layer substantially covering the first coating layer and consisting essentially of c-1) an anionic (meth)acrylate copolymer in an amount of about 15% to about 20% of the total weight of the particle, such as in an amount of about 16% to about 18% of the total weight of the particle, c-2) triethyl citrate (TEC) in an amount of about 1% to about 2% of the total weight of the particle, c-3) glycerol monostearate in an amount of about 0.5% to about 1% of the total weight of the particle, and c-4) polyoxyethylene (20) sorbitan monooleate in an amount of about 0,1% to about 1% of the total weight of the particle, wherein the sum of all ingredients adds up to 100% of the total weight of the particle.

According to certain embodiments, the particle comprises (or consists essentially of): a) a core comprising microcrystalline cellulose, said core comprises about 25% to about 35% of the total weight of the particle; b) a first coating layer substantially covering the core and comprising b-1) lactoferrin in an amount of about 30% to about 50% of the total weight of the particle such as in an amount of about 35% to about 45% of the total weight of the particle, b-2) hydroxypropylmethylcellulose in an amount of about 5% to about 10% of the total weight of the particle, such as in an amount of about 5% to about 8% of the total weight of the particle, and b-3) polyethylene glycol (such as polyethylene glycol having an average molecular weight of 6000 g/mol) in an amount of about 1% to about 2% of the total weight of the particle, such as in an amount of about 1% to about 1.5% of the total weight of the particle; and c) a second coating layer substantially covering the first coating layer and comprising c-1) an anionic (meth)acrylate copolymer in an amount of about 15% to about 20% of the total weight of the particle, such as in an amount of about 16% to about 18% of the total weight of the particle, c-2) triethyl citrate (TEC) in an amount of about 1% to about 2% of the total weight of the particle, and c-3) talc in an amount of about 3% to about 12% of the total weight of the particle, wherein the sum of all ingredients adds up to 100% of the total weight of the particle.

According to certain embodiments, the particle comprises (or consists essentially of): a) a core consisting essentially of microcrystalline cellulose, said core comprises about 25% to about 35% of the total weight of the particle; b) a first coating layer substantially covering the core and consisting essentially of b-1) lactoferrin in an amount of about 30% to about 50% of the total weight of the particle such as in an amount of about 35% to about 45% of the total weight of the particle, b-2) hydroxypropylmethylcellulose in an amount of about 5% to about 10% of the total weight of the particle, such as in an amount of about 5% to about 8% of the total weight of the particle, and b-3) polyethylene glycol (such as polyethylene glycol having an average molecular weight of 6000 g/mol) in an amount of about 1% to about 2% of the total weight of the particle, such as in an amount of about 1% to about 1.5% of the total weight of the particle; and c) a second coating layer substantially covering the first coating layer and consisting essentially of c-1) an anionic (meth)acrylate copolymer in an amount of about 15% to about 20% of the total weight of the particle, such as in an amount of about 16% to about 18% of the total weight of the particle, c-2) triethyl citrate (TEC) in an amount of about 1% to about 2% of the total weight of the particle, and c-3) talc in an amount of about 3% to about 12% of the total weight of the particle, wherein the sum of all ingredients adds up to 100% of the total weight of the particle.

The particle of the present invention may have any suitable form. For example, it may be in the form of a pellet, spheroid or bead. According to some embodiments, the particle is in the form of a pellet.

The particle of the present invention may have one or more (such as all) of the following characteristics:
a) a mean particle size d50 of about 450 μm to about 650 μm;
b) a bulk density of about 0.7 g/ml to about 0.8 g/ml, such as about 0.740 g/mL;
c) a tapped density of about 0.75 g/ml to about 0.85 g/ml, such as about 0.802 g/mL; and
d) a dissolution profile wherein at most 10% of lactoferrin is released from the particle within 120 minutes as measured by an in vitro dissolution test according to Ph. Eur. 2.9.3.

The mean particle size d50 can be determined according to 2.9.31. PARTICLE SIZE ANALYSIS BY LASER LIGHT DIFFRACTION, Ph.Eur. 10.0

The bulk density can be determined according to 2.9.34. BULK DENSITY AND TAPPED DENSITY OF POWDERS, Ph.Eur. 10.0

The tapped density can be determined according to 2.9.34. BULK DENSITY AND TAPPED DENSITY OF POWDERS, Ph.Eur. 10.0

In a related aspect, the present invention provides a pharmaceutical composition comprising one or more (such as a plurality of) particles according to the present invention, and optionally one or more pharmaceutically acceptable excipients.

Suitable pharmaceutically acceptable excipients are well-known to the skilled person and have been described in the literature, such as in Remington's Pharmaceutical Sciences, the Handbook of Pharmaceutical Additives or the Handbook of Pharmaceutical Excipients. Non-limiting examples of suitable pharmaceutically acceptable excipients include diluents, fillers, binding agents, disintegrating agents, lubricants, fluidizers, granulating agents, coating materials, wetting agents, solvents, co-solvents, suspending agents, emulsifying agents, sweetening agents, flavoring agents, odor masking agents, coloring agents, anti-caking agents, chelating agents, plasticisers, viscosifiers, antioxidants, antiseptics, stabilizing agents, surfactants and buffer agents.

In a further related aspect, the present invention provides an oral dosage form comprising one or more (such as a plurality of) particles according to the present invention.

The oral dosage form may be a solid dosage form, such as a capsule or tablet. According to some embodiments, the oral dosage form is a capsule, such as a hard capsule.

The pharmaceutical composition according to the present invention or the oral dosage form according to the present invention may be used as a medicament, such as in the prophylaxis and/or treatment of an intestinal disorder, such as Traveler's diarrhea.

The pharmaceutical composition according to the present invention or the oral dosage form according to the present invention may be used in the regulation of the healthy balance of human intestine microbiota.

In a further related aspect, the present invention provides a food or food supplement comprising one or more (such as a plurality of) particles according to the present invention. The food or food supplement according to the present invention may be used for balancing the ratio between a pathogen and the beneficial microorganisms in the human intestine.

As follows from the above, the multilayered particle of the present invention has an “onion-like” structure and is in particular prepared according to stepwise or continuous coating steps using one or several coating techniques well-known to the skilled person in the art such as layering techniques such as solution layering or suspension layering. Suitable equipment such as coating pan, coating, granulator or fluidized bed coating apparatus using water and/or organic solvents may be used.

Therefore, in a further related aspect, the present invention provides a process for producing the particle according to the present invention, comprising applying the first coating layer in the form of an aqueous coating solution, suspension or dispersion in a spray procedure or by fluidized bed spray granulation onto the core, and applying the second coating layer in the form of an aqueous coating solution, suspension or dispersion in a spray procedure or by fluidized bed spray granulation onto the first coating layer.

The first layer coating may, for example, be applied to the particle core in the form of an aqueous coating solution, suspension or dispersion using fluidized bed technology, such as Wurster coating or rotor coating.

Similarly, The second layer coating may, for example, be applied to the first coating layer in the form of an aqueous coating solution, suspension or dispersion using fluidized bed technology, such as Wurster coating or rotor coating.

One skilled in the art may readily appreciate that the coating conditions, such as solution spray rate, drying air temperature and flow rate must be adjusted in order to achieve an equilibrium between the rate of application of the liquid coating solution, and the rate of evaporation of the solvents such that the first and second coatings can be deposited uniformly on the particle to form a continuous film without overwetting the particle surface. Details of these methods are well-known in the art and set forth in, for example, Lieberman et al., “Pharmaceutical Dosage Forms-Tablets: Volume 3”, Chapter 3: Particle Coating Methods (1990), which is incorporated by reference herein.

As a pre-step, the process may comprise forming the core by direct compression, wet or dry granulation, or direct pelleting of the core-forming material.

The obtained particles according to the present invention may then be formulated either as a pharmaceutical composition or used in the preparation of a food or food supplement. For example, the obtained particles according to the present invention may be filled into a capsule, such as a hard capsule, or compressed into tables, in order to obtain an oral dosage form according to the present invention.

As used herein, the term “about” means plus or minus 10% of the numerical value of the number with which it is being used. Where a numerical limit or range is stated herein, the endpoints are included. Also, all values and sub-ranges within a numerical limit or range are specifically included as if explicitly written out.

As used herein, the indefinite articles “a” and “an” mean “at least one” or “one or more” unless the context clearly dictates otherwise.

As used herein, the terms “comprising”, “including”, “having” and grammatical variants thereof are to be taken as specifying the stated features, steps, or components but do not preclude the addition of one or more additional features, steps, components or groups thereof. The use of “comprising” and “comprises” as used herein is to be understood as also disclosing “consisting essentially of” and “consists essentially of” as well as “consisting of” and “consists of”, respectively.

As used herein, the term “consisting essentially of” (and grammatical variants thereof) generally means that additional materials, features, components, elements or steps may be included that do not materially affect the basic and novel characteristic(s) of the claimed invention. For example, when used in the context of the particle of the invention as well as one of its layers, the term means that the particle or layer may contain additional features, components or elements in addition to those literally disclosed provided that these additional features, components or elements do not materially affect the basic and novel characteristic(s) of the claimed particle. For example, the particle or layer may include additional non-essential elements such as water (e.g., in form of moisture) and/or impurities.

As used herein, the term “substantially covering” (and grammatical variants thereof) means that the coating is generally continuous and generally covers the entire surface of the core or underlying layer, so that little to none of the core or underlying layer is exposed.

Furthermore, it will be understood by the skilled person that the total amount of all ingredients making out the particle or any of its layers will normally add up to 100% of the total weight of the particle or layer, respectively. In other words, the total amount of all ingredients will not exceed 100% of the total weight of the particle or layer.

Having generally described this invention, a further understanding can be obtained by reference to certain specific examples, which are provided herein for purposes of illustration only, and are not intended to be limiting unless otherwise specified.

### EXAMPLES

### 1. Formulation Composition

The formulation is a hard capsule of size 0. The volume of the capsule body is 0.67 mL. Capsules are filled with pellets. Pellets are composed of a core made from microcrystalline cellulose. Cellets 200, which are highly spherical starter pellets consisting of 100% microcrystalline cellulose, have been used for that purpose.

### Lactoferrin Coating Layer

The pellet core has been coated with two layers of coating. The first layer comprising lactoferrin, HPMC (Pharmacoat 606), and polyethylene glycol (PEG 6000).

Lactoferrin has been used in form of a dry powder that contains at least 85% of lactoferrin. The powder is dissolved in purified water before the beginning of the coating process. Alternatively, a chromatographic elution with lactoferrin could be used directly for coating, without the need for drying of the elution and obtaining the lactoferrin powder. Elution that is feasible for use in the coating process should contain from 4-25% of lactoferrin. We have used concentrations 7-16%. Lactoferrin concentration is not fixed but it varies from batch to batch and according to lactoferrin total mass, HPMC and PEG 6000 are added.

HPMC (Pharmacoat 606) is a well-known polymer that is used in pharmaceutical coating processes. 606 is a type of polymer, the degree of polymerization (polymer chain length) determines the viscosity of the HPMC solution.

PEG 6000 is a polymer that decreases the glass transition temperature of the HPMC and makes the coating less fragile at room temperature. PEG 6000 is a plasticizer. Alternatively, one could use a different type of PEG for the same purpose.

The main composition of the lactoferrin layer containing:

TABLE 1
Composition of the lactoferrin layer.

| Compound | W (%)* |
|---|---|
| Lactoferrin | 78.1 |
| PEG 600 | 2.4 |
| Pharmacoat 606 | 13.6 |
| Impurities | 5.9 |

*note that dry coating could contain up to 5% of moisture

### Enteric Coating Layer

Enteric layer is made according to the polymer manufacturer's recipe. The second coating contains a protective polymer (Eudragit® L), glycerol monostearate (GMS), triethyl citrate (TEC) and polysorbate (Tween 80). Enteric coating layer represents 20% of the pellets.

TABLE 2
Enteric layer composition.

| Compound | W (%)* |
|---|---|
| Eudragit ® L | 85.5 |
| TEC | 8.5 |
| GMS | 4.3 |
| Tween 80 | 1.7 |

*note that dry coating could contain up to 5% of moisture

TABLE 3
Alternative composition of enteric coating using talc

| Compound | W (%) |
|---|---|
| Eudragit ® L | 62.5 |
| TEC | 6.3 |
| Talc | 31.2 |

### Pellets Total Composition

The composition of the total formulation is presented in table 4. Composition is defined for 93% pure lactoferrin. The composition could vary, and it is dependent on the purity of the lactoferrin. Therefore the amounts of Pharmacoat 606, PEG 6000, impurities and Cellets 200 are varied.

TABLE 4
Composition of the whole formulation for 93% pure lactoferrin.

| Compound | W (%) | W(Layer) % | Layer |
|---|---|---|---|
| Cellets 200 | 30.3 | 30.3 | Pellet core |
| Lactoferin | 38.8 | 49.7 | Lactoferrin |
| Pharmacoat 606 | 6.8 | | coating |
| PEG 6000 | 1.2 | | |
| Impurities | 2.9 | | |
| Eudragit L | 17.1 | 20.0 | Enteric |
| TEC | 1.7 | | coating |
| GMS | 0.9 | | |
| Tween 80 | 0.3 | | |

* note that dry pellets could contain up to 5% of moisture

### 2. Lactoferrin Coating Using Solid Lactoferrin in Form of a Powder

This example is defined for lactoferrin purity 93% that was the average purity that we have used.
1. Dissolve 417 g of lactoferrin containing powder in 3120 g of purified water to obtain turbid dark red dispersion of lactoferrin.
2. Separately warm up 370 g of purified water to 70° C. and during fast stirring add 11.9 g of PEG 6000 and 67.5 g of Phramacoat 606. Stir until all agglomerates of Pharmacoat 606 disappear and cool the dispersion at room temperature to 30° C.
3. Add dissolved binder solution to a lactoferrin solution and stir to obtain a homogenous coating dispersion.

### 3. Lactoferrin Coating Using Chromatographic Elution of Lactoferrin

For this example, we assume that elution containing lactoferrin contains 10% of total dissolved or undissolved solids from which 93% is lactoferrin and 7% are impurities.
1. Weigh 4170 g of elution containing lactoferrin.
2. Separately warm up 370 g of purified water to 70° C. and during fast stirring add 11.9 g of PEG 6000 and 67.5 g of Phramacoat 606. Stir until all agglomerates of Pharmacoat 606 disappear and cool the dispersion at room temperature to 30° C.
3. Add dissolved binder solution to a lactoferrin solution and stir to obtain a homogenous coating dispersion.

### 4. Coating Process Parameters

Pellets should be coated in the bottom spray fluid bed coater (Glatt GPCG1) using the Wurster chamber. Place 303 g of Cellets 200 in the processing chamber. Start the fluidization process, set up the inlet temperature to 70° C. and heat up the pellets to 38° C. When the temperature is reached start the spraying process and guide the coating at the following conditions:

| | | | | Nozzle | |
|---|---|---|---|---|---|
| Air inlet | Product | Outlet | Air | air | Dispersion |
| temperature | temperature | Temperature | flow | pressure | mass flow |
| (° C.) | (° C.) | (° C.) | (m³/h) | (bar) | (g/min) |
| 70 | 35 | 35 | 61 | 1.8 | 16 |

Spray nozzle diameter should be 1.2 mm and Wurster cylinder position 15 mm above the distribution plate. Coating dispersion should be prepared according to sections 2. and 3. above.

### 5. Enteric Coating

Enteric coating is applied after the finished first layer coating. Prepare the Wurster chamber at the same configuration as in example 3. Weigh 800 g of lactoferrin coated pellets and start the fluidization process. Warm up the pellets to 35° C. and start a spraying process. Guide the coating at the following conditions:

| | | | | Nozzle | |
|---|---|---|---|---|---|
| Air inlet | Product | Outlet | Air | air | Dispersion |
| temperature | temperature | Temperature | flow | pressure | mass flow |
| (° C.) | (° C.) | (° C.) | (m³/h) | (bar) | (g/min) |
| 50 | 30 | 31 | 70 | 1.8 | 8 |

Apply 200 g of the coating dispersion. Coating dispersion should be prepared according to the manufacturer's suggestion (https://corporate.evonik.com/en).

### 6. Capsule Filling

The finished product should be sieved through a sieve with a mesh size of 710 μm to extract all the agglomerates before capsule filling. A hard capsule of size 0 with an hydroxypropylmethylcellulose shell was then filled with particles obtained used. But alternatively, also the gelatin capsules or any other kind of shell could be used.

### 7. Testing

### 7.1 Dissolution Profile

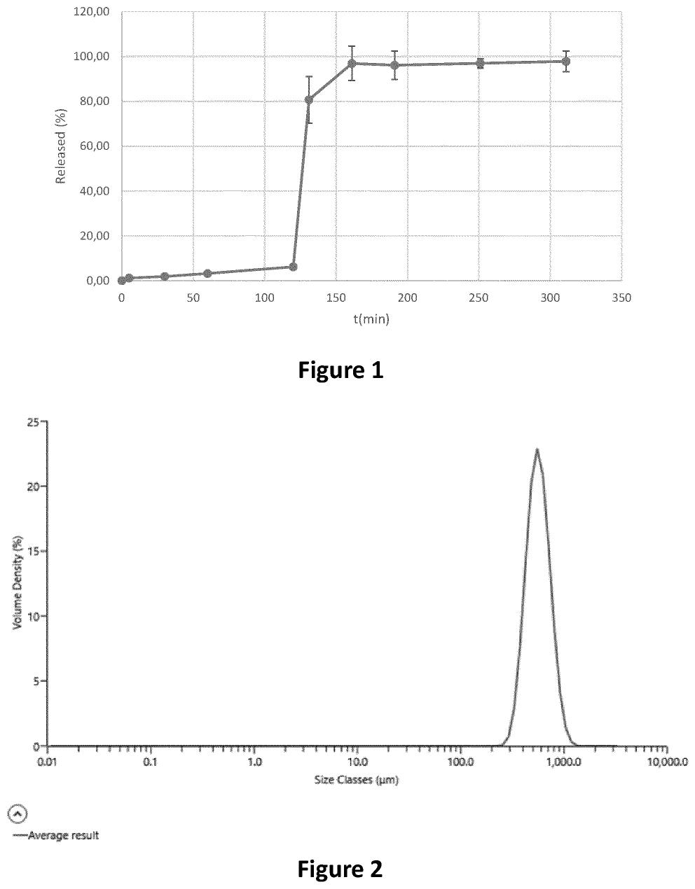

Pellets dissolution profile was tested with “USP II apparatus”, which is generally used standard dissolution method in the field of pharmacy. Dissolution testing was conducted according to pharmacopeia guidelines for enteric coated formulations. Two dissolution media were used. First, the 0.1 M HCl solution was used to simulate the conditions in the stomach for two hours, then Na₃PO₄ solution was added to raise the pH of the medium to pH 6.8. The volume of acidic media was 750 ml and the volume of 0.2 M Na₃PO₄×12H₂O was 250 mL. 10 g of pellets were tested at each run. The temperature of the media was set to 37° C. and the rotation of the paddles to 100 rpm. Dissolution samples were taken at 5, 30, 60, 120, 125, 150, 180, 240 and 300 min. Sampling volume (2.3 mL) was replaced with a fresh phosphate buffer with pH 6.8. All samples were filtered with PVDF 0.2 μm membrane filters and analyzed with HPLC. The dissolution profile (FIG. 1) fits the regulatory criteria which states that less than 10% of the active ingredient should be released after 2 h in the acidic medium. Also, after an increase of pH to 6.8 more than 80% of the total dose was released in less than 30 min.

### 7.2 Particle Size Distribution

Particle size distribution was determined using the Malvern Mastersizer 3000 laser diffraction analyzer. An Aero S dry sample dispersing unit was used. The pellets were transferred to the cone of the dispersing unit, which had a gap position set to the value of 3.0. Air pressure of 1.2 bar and a feed rate of 25% were used. The measurement duration was 4 s, and the background measurement was also set to 4 s. The measurement was performed in five repetitions and the average values are shown in FIG. 2.

### 7.3 Bulk Density

Pellets were carefully transferred to the measuring cylinder. Their mass was weighed and the volume of the pellets was read from the graduated cylinder. Bulk density was calculated based on the obtained results. The process was repeated three times and the density was expressed as an average result.

Bulk density (g/mL)

| | |
|---|---|
| | 0.735 |
| | 0.740 |
| | 0.744 |
| Average: | 0.740 |

### 7.4 Conclusions

The results presented show that the layers of the different coating dispersions are compatible. In addition, the process is highly efficient, with efficiencies of more than 90% achievable on a relatively small scale. A product with a moisture content of less than 7% is produced with only one processing unit. The protective layer has proven essential in extending the shelf life of the formulation. It also facilitates safe intestinal delivery of LF by preventing contact of LF with the harsh gastric environment. The formulation presented here contains a high dose of LF, which has a beneficial effect on the microbiota of the human intestine through a selective bacteriostatic effect on pathogenic microorganisms. The formulation, in the form of a pellet-filled capsule, allows patients to take the capsule with or without food regardless of the time of day. The protective layer effectively protects LF and does not significantly affect the release rate after the pH of the environment is increased. As shown in FIG. 1, the release after 120 minutes matches the immediate release formulations. Consequently, the formulation of the present invention is a sustained release formulation containing LF at a dose suitable for the treatment or prophylaxis of various intestinal disorders, such as Traveler's diarrhea.

1. Enteric-coated particle comprising: a) a core comprising an inert core-forming material selected from cellulose polymer, sugar, sugar alcohol, starch and carnauba wax; b) a first coating layer substantially covering the core and comprising b-1) lactoferrin, b-2) a pharmaceutically acceptable binder and optionally b-3) one or more other suitable excipients, such as a plasticizer; and c) a second coating layer substantially covering the first coating layer and comprising c-1) an enteric coating material, and optionally c-2) one or more suitable excipients, such as a plasticizer and/or an anti-tacking agent.

2. The particle according to claim 1, wherein the inert core-forming material is a cellulose polymer.

3. The particle according to claim 2, wherein the cellulose polymer is selected from the group consisting of microcrystalline cellulose, hydroxyethylcellulose, hydroxypropylcellulose, hydroxypropylmethylcellulose and carboxymethylcellulose.

4. The particle according to claim 2, wherein the cellulose polymer is microcrystalline cellulose.

5. The particle according to any one of claims 1 to 4, wherein the pharmaceutically acceptable binder comprised by the first coating layer is selected from the group consisting of cellulose polymers, including hydroxypropylmethylcellulose, hydroxyethylcellulose, hydroxypropylcellulose and carboxymethylcellulose, povidones, including polyvinylpyrrolidone (PVP), copovidones, agar, gelatin, gummi arabicum, alginates, including sodium alginate and polyetylene glycol alginate, polyethylene glycols, polyvinyl alcohols, sugars, sugar alcohols, starches and modified starches including potato starch, maize starch, rice starch, and pre-gelatinised starch.

6. The particle according to any one of claims 1 to 5, wherein the pharmaceutically acceptable binder comprised by the first coating layer is a non-polyvinylpyrrolidone (PVP) binder.

7. The particle according to any one of claims 1 to 6, wherein the pharmaceutically acceptable binder comprised by the first coating layer is hydroxypropylmethylcellulose.

8. The particle according to any one of claims 1 to 7, wherein the first coating layer comprises a plasticizer.

9. The particle according to claim 8, wherein the plasticizer is selected from the group consisting of polyethylene glycols, polyethylene glycol monomethyl ether, propylene glycol, sorbitol-sorbitansolution, glycerin, deacetylated monoglycerides, tributyl citrate, acetyl tributyl citrate, triethyl citrate, acetyl-triethyl-citrate, castor oil, dibutyl sebacate, diethyl phthalate, and triacetin.

10. The particle according to claim 8, wherein the plasticizer is a polyethylene glycol.

11. The particle according to claim 10, wherein the polyethylene glycol has an average molecular weight of 6000 g/mol.

12. The particle according to any one of claims 1 to 11, wherein the enteric coating material comprised by the second coating layer is selected from polymers or copolymers comprising anionic side groups (preferably carboxylic side groups).

13. The particle according to any one of claims 1 to 12, wherein the enteric coating material comprised by the second coating layer is selected from the group consisting of anionic (meth)acrylate copolymers and anionic polyvinyl polymers or copolymers.

14. The particle according to any one of claims 1 to 13, wherein the enteric coating material comprised by the second coating layer is an anionic (meth)acrylate copolymer.

15. The particle according to any one of claims 1 to 14, wherein the enteric coating material comprised by the second coating layer is an anionic (meth)acrylate copolymer composed of methacrylic acid and methyl methacrylate or ethyl acrylate, or an anionic (meth)acrylate copolymer composed of ethyl acrylate and methyl methacrylate.

16. The particle according to any one of claims 1 to 14, wherein the enteric coating material comprised by the second coating layer is a polymer combination comprising an anionic (meth)acrylate copolymer composed of methacrylic acid and ethyl methacrylate (e.g., EUDRAGIT® L), and an anionic (meth)acrylate copolymer composed of ethyl acrylate and methyl methacrylate (e.g., EUDRAGIT® NM), preferably in the ratio of 25:75.

17. The particle according to any one of claims 1 to 16, wherein the second coating layer further comprises a plasticizer.

18. The particle according to claim 17, wherein the plasticizer is selected from the group consisting of polyethylene glycols, polyethylene glycol monomethyl ether, propylene glycol, sorbitol-sorbitan solution, glycerin, deacetylated monoglycerides, tributyl citrate, acetyl tributyl citrate, triethyl citrate, acetyl-triethyl-citrate, castor oil, dibutyl sebacate, diethyl phthalate, and triacetin.

19. The particle according to claim 17 or 18, wherein the plasticizer is triethyl citrate (TEC).

20. The particle according to any one of claims 1 to 19, wherein the second coating layer further comprises an anti-tacking agent.

21. The particle according to claim 20, wherein the anti-tacking agent is talc or glycerol monostearate.

22. The particle according to any one of claims 1 to 21, wherein the second coating layer further comprises an emulsifier.

23. The particle according to claim 22, wherein the emulsifier is a non-ionic emulsifier.

24. The particle according to claim 23, wherein the non-ionic emulsifier is a polysorbate.

25. The particle according to claim 24, wherein the polysorbate is polyoxyethylene (20) sorbitan monooleate.

26. The particle according to claim 1, comprising: a) a core comprising microcrystalline cellulose; b) a first coating layer substantially covering the core and comprising b-1) lactoferrin, b-2) hydroxypropylmethylcellulose and b-3) polyethylene glycol; and c) a second coating layer substantially covering the first coating layer and comprising c-1) an anionic (meth)acrylate copolymer, c-2) triethyl citrate (TEC), c-3) glycerol monostearate, and c-4) polyoxyethylene (20) sorbitan monooleate.

27. The particle according to claim 1, comprising: a) a core comprising microcrystalline cellulose; b) a first coating layer substantially covering the core and comprising b-1) lactoferrin, b-2) hydroxypropylmethylcellulose and b-3) polyethylene glycol; and c) a second coating layer substantially covering the first coating layer and comprising c-1) an anionic (meth)acrylate copolymer, c-2) triethyl citrate (TEC) and c-3) talc.

28. Pharmaceutical composition comprising one or more particles according to any one of claims 1 to 27, and optionally one or more pharmaceutically acceptable excipients.

29. Oral dosage form comprising one or more particles according to any one of claims 1 to 27.

30. Oral dosage form according to claim 29, which is a hard capsule.

31. The oral dosage form according to any of claims 29-30, which comprises 200 mg or more of lactoferrin.

32. The oral dosage form according to any of claims 29-31, which comprises from about 400 mg to about 600 mg of said particles.

33. The particle according to any one of claims 1 to 27, the pharmaceutical composition according to claim 28 or the oral dosage form according to any one of claims 29 to 32 for use as a medicament, preferably for use in the prophylaxis and/or treatment of an intestinal disorder.

34. Food or food supplement comprising one or more particles according to any one of claims 1 to 27.

35. Process for producing the particle according to any one of claims 1 to 27, comprising applying the first coating layer in the form of an aqueous coating solution, suspension or dispersion in a spray procedure or by fluidized bed spray granulation onto the core, and applying the second coating layer in the form of an aqueous coating solution, suspension or dispersion in a spray procedure or by fluidized bed spray granulation onto the first coating layer.
